(19) United States
(12) Patent Application Publication
(10) Pub. No.: US 2007/0196279 A1
(43) Pub. Date: Aug. 23, 2007

(54) Methods for treating progressive neurodegenerative disorders

(76) Inventors: Che-Kun James Shen, Taipei-City (TW); Kuen-Jer Tsai, Kaohsiung City (TW)

Correspondence Address: OCCHIUTI ROHLICEK & TSAO, LLP, 10 FAWCETT STREET, CAMBRIDGE, MA, 02138, US

(21) Appl. No.: 11/358,392

(22) Filed: Feb. 21, 2006

(51) Int. Cl. A61K 49/00; A61K 38/19; A61K 38/17; A01K 67/027

(52) U.S. Cl. 424/9.2; 514/12; 424/85.1; 800/3; 800/12

(57) ABSTRACT

Disclosed herein is a method for treating a progressive neurodegenerative disorder by systemically administering a composition containing an effective amount of a G-CSF receptor agonist. The composition can also be administered to inhibit the onset of a progressive neurodegenerative disorder in a subject at high risk thereof. A further method relates to selecting a G-CSF receptor agonist of high efficacy for treating a progressive neurodegenerative disorder.

### BACKGROUND

Progressive neurodegenerative disorders (PNDs), exemplified by Alzheimer's Disease, cause a slow but inexorable loss of neurons that is accompanied by degrading cognitive or motor function and is followed by death of the afflicted individual. The effects of PNDs are devastating to the quality of life of those afflicted as well as that of their families. Moreover, PNDs impose an enormous health care burden on society. Indeed, as this class of diseases primarily affects the expanding elderly population, their prevalence and societal impact are expected to become even more severe in the coming years.

One of the most promising therapeutic approaches for treating PNDs is neuronal replacement with transplanted neurons derived from stem cells, which are found scattered throughout various tissues of the adult human body in very small numbers. Human embryonic stem cells (HESCs) are the most well characterized for potential therapeutic applications. Unfortunately, the development of HESC lines in sufficient quantity and of adequate quality for clinical applications has been severely hampered by controversy over their embryonic origin. However, even if clinical-grade HESC lines do become readily available, transplanting in vitro-differentiated, HESC-derived neurons is risky and requires highly invasive intracerebral injection of the neurons into a patient. Thus, there is an urgent and ongoing need for methods that afford low risk, non-invasive replenishment of neurons for treating PNDs or inhibiting their onset.

### SUMMARY

The present invention is based, in part, on the finding that granulocyte-colony stimulating factor receptor (G-CSFR) agonists, when systemically administered to a subject suffering from a PND, stimulate the migration of endogenous stem cells from bone marrow into degenerating brain regions (e.g., hippocampus and cortex), where they promote increased neurogenesis.

Accordingly, one aspect of the invention relates to treatment of a PND by systemically administering a composition containing an effective amount of a G-CSFR agonist to a subject suffering from the PND. The systemically administered G-CSFR agonist thereby mobilizes hemopoetic stem cells from bone marrow into peripheral blood. The stem cells circulating in peripheral blood are then able to cross the blood brain barrier into degenerating brain regions.

A G-CSFR agonist refers to a molecule that binds to and activates a G-CSFR, e.g., G-CSF itself, G-CSF sequence-related variants, G-CSFR agonist monoclonal antibodies or antibody-derived polypeptides, or small molecule compounds.

A PND includes any condition that leads to neuronal cell death over a period greater than 3 days (e.g., one month or 20 years) and are behaviorally manifested as abnormal and worsening cognitive abilities or motor functions in an afflicted subject. Preferably, prior to administration of the composition, the subject is diagnosed as having a PND. PNDs include those that decrease a cognitive ability (e.g., short term memory, long term memory, spatial orientation, face recognition, or language ability). Some PNDs result from hippocampal neurodegeneration (at least in part). Some examples of a PND are Alzheimer's disease, Parkinson's disease, Huntington's disease, Lewy body dementia, or Pick's disease. Of note, some PNDs affect both cognitive abilities and motor functions (e.g., Huntington's disease).

Another aspect of the invention relates to inhibiting the onset of a PND. A subject at high risk of developing a PND, e.g., a subject diagnosed as such, is systemically administered a composition containing an effective amount of a G-CSFR agonist.

A further aspect of the invention relates to a method for selecting a G-CSFR agonist of high efficacy for treating a PND. In the method, a G-CSFR agonist is systemically administered to a non-human test mammal suffering from a PND. The test animal's performance in a behavioral task is then determined and compared to that of a control mammal suffering from the same PND, but not administered a G-CSFR agonist. Better performance by the test mammal than by the control mammal indicates that the G-CSFR agonist is of high efficacy for treating the PND.

The test mammal and control mammal can be genetically modified, e.g., to overexpress a transgene so that they develop a PND. Alternatively, a PND that impairs learning or memory can be induced by administering aggregated amyloid β (Aβ) peptide to the test mammal and the control mammal.

Other features or advantages of the present invention will be apparent from the following detailed description, and also from the claims.

### DETAILED DESCRIPTION

Methods are described for treating a subject suffering from a PND. The methods include systemic administration of a composition containing an effective amount of a G-CSFR agonist to the subject. The G-CSFR agonist thus administered induces the migration of hemopoetic stem cells from bone marrow to degenerating brain regions where the stem cells promote increased neurogenesis. The G-CSFR agonist can also be administered to a subject at high risk of a PND as a method of inhibiting its onset. Also described are methods for selecting a G-CSFR agonist of high efficacy for treating a PND by testing its efficacy in non-human mammals suffering from a PND.

A G-CSFR agonist can be a purified mammalian polypeptide that includes the amino acid sequence of a mature mammalian G-CSF (e.g., human, mouse, or rat G-CSF), namely, one that does not include a signal peptide sequence. For example, the G-CSFR agonist can include amino acids 13-186 of human G-CSF (GenBank Accession No. AAA03056):

(SEQ ID NO:1)

TPLGPASSLPQSFLLKCLEQVRKIQGDGAALQEKLCATYKLCHPEELVLL
GHSLGIPWAPLSSCPSQALQLAGCLSQLHSGLFLYQGLLQALEGISPELG
PTLDTLQLDVADFATTIWQQMEELGMAPALQPTQGAMPAFASAFQRRAGG
VLVASHLQSFLEVSYRVLRHLAQP

A mammalian G-CSF or G-CSF-containing polypeptide can be purified using standard techniques from a native source (e.g., a cell line that secretes native G-CSF) or a recombinant expression source (e.g., E. coli, Yeast, insect cells, or mammalian cells that express transgenic G-CSF). Recombinant human G-CSF can also be purchased from a commercial source, e.g., Amgen Biologicals (Thousand Oaks, Calif.). Alternatively, recombinant G-CSF can be purified as described in, e.g., U.S. Pat. No. 5,849,883.

The G-CSFR agonist can be a G-CSF sequence variant (as described in, e.g., U.S. Pat. Nos. 6,358,505 and 6,632,426) that is at least 70% identical to SEQ ID NO:1 (i.e., having any percent identity between 70% and 100%). In general, G-CSF sequence variations should not alter residues critical to G-CSF function, including (in human G-CSF) residues K16, E19, Q20, R22, K23, D27, D109, and F144. See, e.g., Young et al., id. and also U.S. Pat. No. 6,358,505, example 29.

When comparing a G-CSF sequence with that of a sequence variant, the percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. The percent identity between two amino acid sequences can be determined using the Needleman and Wunsch (1970), J. Mol. Biol. 48:444-453, algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The G-CSFR agonist can be a chemically modified mammalian G-CSF, e.g., one having a linked polyethylene glycol moiety as described in U.S. Pat. No. 5,824,778.

Alternatively, the G-CSFR agonist can be a monoclonal antibody or antibody-derived molecule (e.g., an Fab fragment) that binds to and activates a G-CSFR as described in, e.g., U.S. Patent Application No. 20030170237.

Preferably, the G-CSFR agonist has a 50% effective concentration (EC50) no greater than about ten times that of G-CSF. In addition, the affinity of the G-CSFR agonist should be no less than about one tenth that of G-CSF. Assays for determining G-CSFR agonist properties are described in detail in, e.g., Young et al. (1997), Protein Science 6:1228-1236 and U.S. Pat. No. 6,790,628. Moreover, such assays can be used to identify entirely novel G-CSFR agonists (e.g., small molecule agonists) that meet the above-mentioned criteria.

The above-described G-CSFR agonists can be used to treat a subject suffering from a PND that decreases a cognitive ability. Examples of PNDs that affect at least one cognitive ability include but are not limited to AD, Parkinson's disease, Huntington's disease, Lewy body Dementia, or Pick's disease. The PND is treated by systemically administering to an afflicted subject a composition containing an effective amount of one the above-described G-CSFR agonists. Prior to administration of the inhibitor composition, the subject can be diagnosed as suffering from a PND. In the case of a disorder that affects a cognitive ability, a subject can be diagnosed by any one of a number of standardized cognitive assays, e.g., the Mini-Mental State Examination, the Blessed Information Memory Concentration assay, or the Functional Activity Questionnaire. See, e.g., Adelman et al. (2005), Am. Family Physician, 71(9):1745-1750. Indeed, in some cases a subject can also be diagnosed as having a high risk of developing a PND, even in the absence of overt symptoms. For example, the risk of Alzheimer's disease in a subject can be determined by detecting a decrease in the volumes of the subject's hippocampus and amygdale, using magnetic resonance imaging. See, e.g., den Heijer et al. (2006), Arch. Gen. Psychiatry, 63(1):57-62. Accordingly, the subject's risk of a PND can be reduced by prophylactically administering to the subject a composition containing an effective amount of a G-CSFR agonist.

G-CSFR agonists of high efficacy for treating a PND can be selected based on their evaluation in a non-human mammal suffering from a PND. The G-CSFR agonist to be tested is systemically administered to a test mammal suffering from a PND known to impair performance of a behavioral task. The test mammal's performance of the task is then assessed and compared to that of a control mammal suffering from the same PND, but not administered the G-CSFR agonist. A better performance by the test mammal indicates that the G-CSFR agonist has high efficacy for treating the PND.

The non-human mammals used in the behavioral task can be, e.g., rodents such as mice, rats, or guinea pigs. Non-rodent species can also be used, e.g., rabbits, cats, or monkeys. In some cases, the non-human mammals are genetically modified to develop a PND. For example they can express a transgene or have suppressed expression of a native gene. Expression of the transgene or suppression of the native gene can be temporally or regionally regulated. Methods for transgene expression and gene suppression as well as their spatial and temporal control in non-human mammals (e.g., in mice and other rodents) are well established. See, e.g, Si-Hoe et al. (2001), Mol Biotechnol., 17(2):151-182; Ristevski (2005), Mol. Biotechnol., 29(2):153-163; and Deglon et al. (2005), J. Gene Med., 7(5):530-539.

A number of transgenic mouse models of PNDs (e.g., Alzheimer's disease, and amylotrophic lateral sclerosis) have been established. See, e.g., Spires et al. (2005), NeuroRx., 2(3):447-64 and Wong et al. (2002), Nat. Neurosci., 5(7):633-639. Such transgenic animal models spontaneously develop a PND that is manifested behaviorally by impaired learning, memory, or locomotion. Such animal models are suitable for selecting high efficacy G-CSFR agonists as described above.

A PND can also be induced in a non-human mammal by non-genetic means. For example, a PND that affects learning and memory can be induced in a rodent by injecting aggregated Aβ peptide intracereberally as described in, e.g., Yan et al. (2001), Br. J. Pharmacol., 133(1):89-96.

Cognitive abilities, as well as motor functions in non-human animals suffering from a PND, can be assessed using a number of behavioral tasks. Well-established sensitive learning and memory assays include the Morris Water Maze (MWM), context-dependent fear conditioning, cued-fear conditioning, and context-dependent discrimination. See, e.g., Anger (1991), Neurotoxicology, 12(3):403-413. Examples of of motor behavior/function assays, include the rotorod test, treadmill running, and general assessment of locomotion.

The above-mentioned G-CSFR agonists can be incorporated into pharmaceutical compositions for prophylactic or therapeutic use. For example, a pharmaceutical composition can include an effective amount of recombinant human G-CSF and a pharmaceutically acceptable carrier. The term “an effective amount” refers to the amount of an active composition that is required to confer a prophylactic or therapeutic effect on the treated subject. Generally, the effective dose will result in a circulating G-CSFR agonist concentration sufficient to reliably increase the numbers of hemapoietic progenitor cells in circulating blood. Nonetheless, effective doses will vary, as recognized by those skilled in the art, depending on the types of PNDs treated and their severity, the stage of intervention, the general health or age of the subject, previous treatments, route of administration, excipient usage, and the possibility of co-usage with other prophylactic or therapeutic treatment.

To practice the methods of the present invention, a G-CSFR agonist-containing composition can be administered systemically via a parenteral or rectal route. The term “parenteral” as used herein refers to subcutaneous, intracutaneous, intravenous, intramuscular, intra-articular, intra-arterial, intrasynovial, intrasternal, intrathecal, or intralesional, as well as any suitable infusion technique.

When administered, the therapeutic composition is preferably in the form of a pyrogen-free, parenterally acceptable aqueous solution. The preparation of such a parenterally acceptable protein solution, having due regard to pH, isotonicity, stability and the like, is within the skill of the art. Among the parentarally acceptable vehicles and solvents that can be employed are mannitol, water, Ringer's solution, and isotonic sodium chloride solution.

As PNDs are chronic conditions, continuous systemic administration is useful for treating an afflicted subject. Methods for continually infusing a composition and sustaining its systemic concentration over time are known in the art. For example, the compositions described herein can be released or delivered from an osmotic mini-pump or other time-release device. The release rate from an elementary osmotic mini-pump can be modulated with a microporous, fast-response gel disposed in the release orifice. An osmotic mini-pump is useful for controlling release of the composition over an extended period of time (e.g., from one week to five months). Such mini pumps as well as other sustained release devices are available commercially from, e.g., DURECT corporation (Cupertino, Calif.). An active composition can also be administered in the form of suppositories for rectal administration.

The following specific example is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE

### G-CSF Rescues Learning Deficits in a Mouse Model of Alzheimer's Disease

An Alzheimer's disease-like PND was induced in mice by intraventricular injection of aggregated Aβ peptide as described in Yan et al., ibid.

Aggregated Aβ was prepared from solutions of 10 mM soluble Aβ₍₁₋₄₂₎ in 0.01 M phosphate-buffered saline, pH 7.4. Aβ peptide was purchased from Sigma-Aldrich (St. Louis, Mo.). The Aβ solution was then incubated at 37° C. for three days to form the aggregated Aβ and stored at −70° C. prior to use. Prior to injection of the aggregated Aβ, eight-week old C57BL/6 male mice were anesthetized by intraperitoneal administration of sodium pentobarbital (40 mg/kg). The aggregated Aβ was then stereotaxically injected into dorsal hippocampus and cortex bilaterally using a 26-gauge needle connected to a Hamilton microsyringe (Hamilton, Reno, Nev.). The injection volume of aggregated Aβ or phosphate buffered saline (PBS; a control solution) was one microliter. After the injection, the resulting PND was allowed to develop over a period of seven days before the mice were assessed for pathology or behavioral deficits. Brain immunohistochemistry was used to confirm that Aβ aggregates formed at the injected sites.

Spatial learning ability of the mice was assessed in the Morris water-maze learning task. The animals were subjected to four trials per session, and two sessions per day, with one session given in the morning and the other in the afternoon. A total of six sessions were given for evaluating the animals. In each of the four trials, the animals were randomly placed at four different starting positions equally spaced around the perimeter of a pool filled with water made opaque by addition of powdered milk. They were then allowed to search for a hidden platform under the surface of the pool. If an animal could not find the platform after 120 seconds, it was guided to the platform. After mounting the platform, the animals were allowed to stay there for 20 seconds. The time required for each animal to find the platform was recorded as the escape latency.

Aβ-treated mice were tested in the Morris water maze spatial learning task and their performance was compared to that of control mice injected with PBS alone. The performance of the Aβ-treated mice was significantly worse than that of the control mice, as demonstrated by a significantly higher escape latency.

Subsequently, the Aβ-treated mice were divided into a G-CSF group and a control control group. Mice in the G-CSF group were injected subcutaneously with recombinant human G-CSF (Amgen Biologicals) at a dose (50 μg/kg) once daily for five days. In parallel, mice in the control group were injected subcutaneously with PBS. Afterwards, the mice from both groups were tested in the water maze task and their performance was compared with that of mice treated with either G-CSF or PBS alone.

Aβ-treated mice in the G-CSF group were found to perform this task significantly better than the mice in the Aβ-treated control group, as demonstrated by an escape latency similar to that of mice treated with either G-CSF or PBS alone.

Consistent with the behavioral rescue by G-CSF, neurogenesis, as assessed by BrdU (a marker of cell proliferation) plus MAP2 (a neuron-specific marker) co-labeling of new neurons, was found to be higher in the cortex and hippocampus of Aβ-treated animals that were administered G-CSF versus the same areas in Aβ-treated animals administered only PBS.

These studies indicated that systemically administered G-CSF could rescue behavioral deficits caused by intracerebral injection of aggregated Aβ and stimulated increased neurogenesis in the injected regions.

### OTHER EMBODIMENTS

All of the features disclosed in this specification may be combined in any combination. Each feature disclosed in this specification may be replaced by an alternative feature serving the same, equivalent, or similar purpose. For example, rather than directly administering a G-CSFR agonist, G-CSF levels in a subject suffering from a PND can be increased by stimulating endogenous production, e.g., by administering an adenosine Al receptor agonist to the subject as described, e.g., in U.S. Pat. No. 6,790,839. Indeed, the use of such compositions is also within the scope of the invention.

From the above description, one skilled in the art can easily ascertain the essential characteristics of the present invention, and without departing from the spirit and scope thereof, can make various changes and modifications of the invention to adapt it to various usages and conditions. Thus, other embodiments are also contemplated.

1. A method for treating a progressive neurodegenerative disorder in a subject in need thereof, the method comprising systemically administering a composition containing an effective amount of a G-CSF receptor agonist to the subject, whereby hemopoetic stem cells are mobilized from bone marrow into peripheral blood.

2. The method of claim 1, wherein the subject is diagnosed as suffering from the progressive neurodegenerative disorder prior to the administration.

3. The method of claim 2, wherein the progressive neurodegenerative disorder decreases a cognitive ability.

4. The method of claim 3, wherein the cognitive ability is memory.

5. The method of claim 3, wherein the progressive neurodegenerative disorder is Alzheimer's disease, Parkinson's disease, Huntington's disease, Lewy body dementia, or Pick's disease.

6. The method of claim 5, wherein the neurodegenerative disorder is Alzheimer's disease.

7. The method of claim 2, wherein the progressive neurodegenerative disorder is caused by hippocampal neurodegeneration.

8. A method for inhibiting the onset of a progressive neurodegenerative disorder in a subject at high risk thereof, the method comprising systemically administering a composition containing an effective amount of a G-CSF receptor agonist to the subject, whereby hemopoetic stem cells are mobilized from bone marrow into peripheral blood.

9. The method of claim 8, wherein the subject is identified as having a high risk of developing the progressive neurodegenerative disorder prior to the administration.

10. The method of claim 9, wherein the progressive neurodegenerative disorder decreases a cognitive ability.

11. The method of claim 10, wherein the cognitive ability is memory.

12. The method of claim 10, wherein the progressive neurodegenerative disorder is Alzheimer's disease, Parkinson's disease, Huntington's disease, Lewy body Dementia, or Pick's disease.

13. The method of claim 12, wherein the progressive neurodegenerative disorder is Alzheimer's disease.

14. The method of claim 2, wherein the progressive neurodegenerative disorder is caused by hippocampal neurodegeneration.

15-22. (canceled)

23. The method of claim 1, wherein the G-CSF receptor agonist is an antibody that binds to and activates a G-CSF receptor.

24. The method of claim 1, wherein the G-CSF receptor agonist the G-CSF receptor agonist includes a G-CSF sequence or a variant thereof.

25. The method of claim 24, wherein the G-CSF sequence includes SEQ ID NO: 1 and the variant is at least 70% identical to SEQ ID NO: 1.

26. A method for promoting neurogenesis in a subject in need thereof, the method comprising systemically administering a composition containing an effective amount of a G-CSF receptor agonist to the subject, whereby hemopoetic stem cells are mobilized from bone marrow into peripheral blood.

27. The method of claim 26, wherein the subject has a progressive neurodegenerative disorder or a high risk of having the disorder.
